(19) United States
(12) Patent Application Publication
(10) Pub. No.: US 2023/0225742 A1
(43) Pub. Date: Jul. 20, 2023

(54) CLIP SYSTEM

(71) Applicants: Masaru Yuasa, Hachioji-shi (JP); Rintaro Haraguchi, Akishima-shi (JP); Shogo Shindo, Hachioji-shi (JP); Yuki Tanaka, Hino-shi (JP); Naoki Takizawa, Musashino-shi (JP); Mayo Kamagata, Machida-shi (JP); Kyosuke Sato, Hachioji-shi (JP); Yasuo Funakoshi, Hachioji-shi (JP); Kensuke Uesaka, Hino-shi (JP); Shoichiro Kawayoke, Hino-shi (JP)

(72) Inventors: Masaru Yuasa, Hachioji-shi (JP); Rintaro Haraguchi, Akishima-shi (JP); Shogo Shindo, Hachioji-shi (JP); Yuki Tanaka, Hino-shi (JP); Naoki Takizawa, Musashino-shi (JP); Mayo Kamagata, Machida-shi (JP); Kyosuke Sato, Hachioji-shi (JP); Yasuo Funakoshi, Hachioji-shi (JP); Kensuke Uesaka, Hino-shi (JP); Shoichiro Kawayoke, Hino-shi (JP)

(21) Appl. No.: 18/156,020

(22) Filed: Jan. 18, 2023

Related U.S. Application Data
(60) Provisional application No. 63/301,121, filed on Jan. 20, 2022.

(51) Int. Cl. A61B 17/128

(52) U.S. Cl. CPC A61B 17/1285; A61B 2017/12004

(57) ABSTRACT

The present invention provides a clip system that is optimal for suture treatment of living tissue. The clip system includes a clip unit having a first arm and a second arm capable of opening and closing, a wire capable of opening and closing the clip, and a sheath through which the wire can be inserted. The clip unit includes a support portion that swingably supports the first arm and the second arm.

### CROSS-REFERENCE TO RELATED APPLICATIONS

The present application claims priority based on U.S. Pat. Provisional Application No. 63/301,121 provisionally filed in the United States on Jan. 20, 2022, the contents of which are incorporated herein by reference.

### BACKGROUND

### Technical Field

The present invention relates to a clip system.

### Background Art

In endoscopic treatment, medical instruments such as clip units are used to treat living tissue. These medical instruments are introduced to the treatment site by means of a device that can be passed through a channel of an endoscope.

Japanese Unexamined Patent Application, First Publication No. H9-38093 (hereinafter referred to as Patent Document 1) describes a treatment tool used to puncture a living tissue.

The treatment instrument described in Patent Document 1 can be left in the body while being punctured into living tissue.

However, the treatment tool described in Patent Document 1 is not necessarily the optimum treatment tool for performing, for example, suturing a tissue resection or a defect.

### SUMMARY

The present invention provides an optimal clip system for suture treatment of living tissue.

A clip system according to a first aspect of the present invention includes a clip unit having a first arm and a second arm capable of opening and closing, a wire capable of opening and closing the clip, and a sheath through which the wire can be inserted. The clip unit includes a support portion that swingably supports the first arm and the second arm.

The clip system of the present invention can suitably perform, for example, suturing an excision or defect.

### BRIEF DESCRIPTION OF THE DRAWINGS

FIG. 1 is a perspective view of an applicator.

FIG. 2 is a perspective view of a clip unit according to a first embodiment.

FIG. 3 is a perspective view of the clip unit with a pair of arms swinging.

FIG. 4 is a perspective view of the clip unit loaded in the applicator.

FIG. 5 is a diagram showing a defect formed in a living tissue inside the body.

FIG. 6 is a cross-sectional view of the defect in a sewing direction.

FIG. 7 is a view showing the clip unit used to treat the defect by a front approach.

FIG. 8 shows the clip unit in use treating the defect with a tangential approach.

FIG. 9 is a diagram showing the clip unit with a pair of arms swinging.

FIG. 10 is a diagram showing a pulling step using the clip unit.

FIG. 11 is a diagram showing a separation step using the clip unit.

FIG. 12 is a diagram showing a modification of the sheath of the applicator.

FIG. 13 is a diagram showing operation of a modified example of the sheath.

FIG. 14 is a diagram showing operation of a modified example of the sheath.

FIG. 15 shows an approach step using a modification of the sheath.

FIG. 16 is a perspective view of a clip unit according to a second embodiment.

FIG. 17 is a side view of a tissue-grasping portion of the clip unit.

FIG. 18 is a perspective view of a pair of arms that grasp a first mucous membrane and a second mucous membrane.

FIG. 19 is a side view of the pair of arms that grasp the first mucous membrane and the second mucous membrane.

FIG. 20 is a diagram showing a pulling step using the clip unit.

FIG. 21 is a perspective view of a clip unit according to a third embodiment.

FIG. 22 is a side view of a tissue-grasping portion of the clip unit.

FIG. 23 is a perspective view of a pair of arms that grasp the first mucous membrane and the second mucous membrane.

FIG. 24 is a perspective view of a clip unit according to a fourth embodiment.

FIG. 25 is a diagram showing an applicator loaded with the clip unit.

FIG. 26 is a diagram showing a first grasping step using the clip unit.

FIG. 27 is a diagram showing the first grasping step using the clip unit.

FIG. 28 is a diagram showing a second grasping step using the clip unit.

FIG. 29 is a diagram showing a separation step using the clip unit.

FIG. 30 is a perspective view of a clip unit according to a fifth embodiment.

FIG. 31 is a diagram showing the first grasping step using the clip unit.

FIG. 32 is a diagram showing the second grasping step using the clip unit.

FIG. 33 is a diagram showing the separation step using the clip unit.

FIG. 34 is a diagram showing another aspect of the first grasping step and the second grasping step using the clip unit.

FIG. 35 is a perspective view of a clip unit according to a sixth embodiment.

FIG. 36 is a diagram showing the first grasping step using the clip unit.

FIG. 37 is a diagram showing the second grasping step using the clip unit.

FIG. 38 is a diagram showing a pulling step using the clip unit.

FIG. 39 is a diagram showing the separation step using the clip unit.

FIG. 40 is a perspective view of a clip unit according to a seventh embodiment.

FIG. 41 is a diagram showing the first grasping step using the clip unit.

FIG. 42 is a diagram showing a first separation step using the clip unit.

FIG. 43 is a diagram showing a connecting step using the clip unit.

FIG. 44 is a diagram showing the second grasping step using the clip unit.

FIG. 45 is a diagram showing a second separation step using the clip unit

FIG. 46 is a diagram showing a clip unit and an applicator according to an eighth embodiment.

FIG. 47 is a view showing actions of a first arm and a second arm of the clip unit.

FIG. 48 is a diagram showing an approach step using the clip unit.

FIG. 49 is a diagram showing a grasping step using the clip unit.

FIG. 50 is a diagram showing a grasping step using the clip unit.

FIG. 51 is a diagram showing a pulling step using the clip unit.

FIG. 52 is a diagram showing a front approach using the clip unit.

FIG. 53 is a diagram showing a tangential approach using the clip unit.

### DETAILED DESCRIPTION OF THE PREFERRED EMBODIMENTS

### First Embodiment

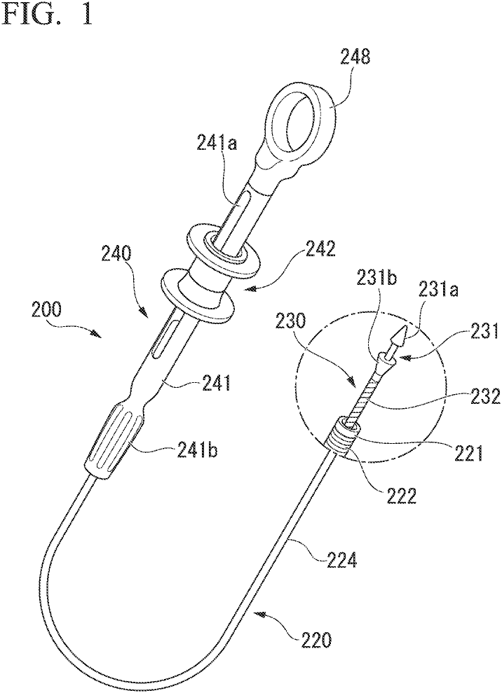

A first embodiment of the present invention will be described with reference to FIGS. 1 to 12.

A clip system (clip device, endoscope suturing device) 300 according to this embodiment includes a clip unit 100 and an applicator 200. The clip unit 100 is loaded into the applicator 200 that can be inserted through a channel of an endoscope and introduced to a treatment position.

### Applicator 200

FIG. 1 is a perspective view of the applicator 200.

The applicator (clip unit introduction device) 200 includes a sheath 220, an operation wire 230, and an operation portion 240. The applicator 200 is inserted through, for example, a treatment instrument insertion channel of an endoscope and used in combination with the endoscope. Therefore, the sheath 220 is formed sufficiently longer than the length of the treatment instrument insertion channel of the endoscope. The sheath 220 is flexible and curves according to the curvature of the insertion portion of the endoscope.

The sheath 220 includes a distal tip 221, a distal coil 222, and a proximal coil 224, and is formed in an elongated tubular shape as a whole. The distal coil 222 is arranged on the distal side of the sheath 220. The distal tip 221 is arranged at the distal end of the distal coil 222.

The operation wire (power transmission member) 230 includes, as shown in FIG. 1, an arrowhead hook portion (connection portion) 231 connected to the clip unit 100, and a wire 232 that operates the arrowhead hook portion 231.

The arrowhead hook portion 231 includes a substantially conical engaging portion 231a that engages with the clip unit 100, and a wire connecting portion 231b provided at the proximal end of the engaging portion 231a. The arrowhead hook portion 231 is made of, for example, a metal material such as stainless steel.

The wire 232 is inserted through the sheath 220 so that it can move back and forth. The distal end of the wire 232 is fixed to the proximal end of the wire connecting portion 231b by welding, for example.

The operation portion 240 includes an operation portion main body 241, a slider 242, and a thumb ring 248, as shown in FIG. 1. The operation portion main body 241, the slider 242 and the thumb ring 248 are injection molded, for example, from a resin material. The operation portion main body 241 includes a slit portion 241a and a rotary grip 241b on the distal side. The slit portion 241a supports the slider 242 so as to be capable of advancing and retracting.

The slider 242 is attached so as to be capable of moving back and forth in the longitudinal axis direction of the operation portion main body 241. and the proximal end of the wire 232 is attached. As the slider 242 advances and retracts along the operation portion main body 241, the wire 232 advances and retracts with respect to the sheath 220, and the arrowhead hook portion 231 advances and retracts.

The thumb ring 248 is attached to the proximal end of the operation portion main body 241 so as to be rotatable around the longitudinal axis of the operation portion main body 241.

### Clip Unit 100

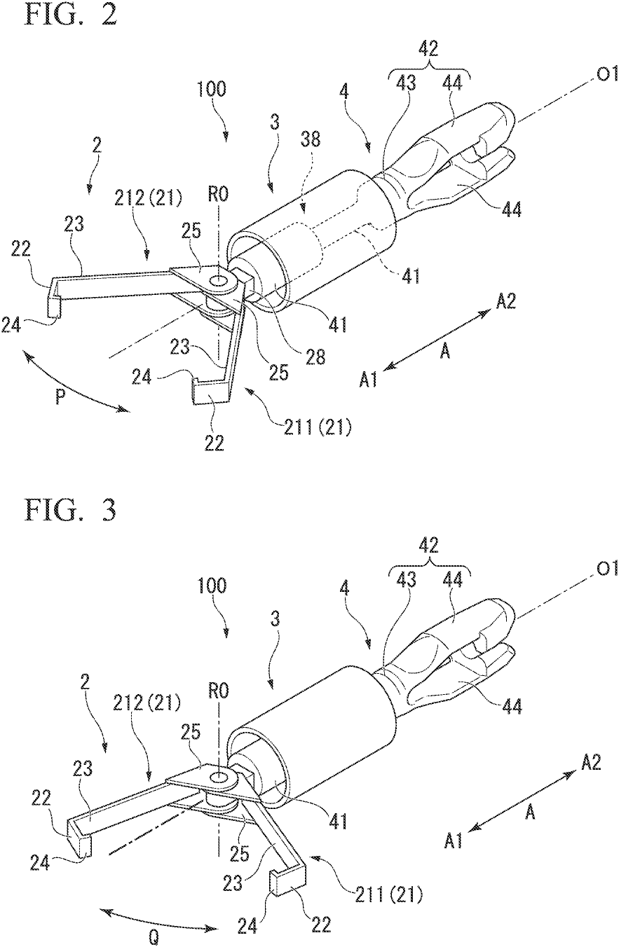

FIG. 2 is a perspective view of the clip unit 100 according to this embodiment.

The clip unit 100 includes a clip 2. a holding tube 3, and a connecting member 4. In the following description, the clip 2 side in the longitudinal direction A of the clip unit 100 is referred to as the distal side (distal side) A1 of the clip unit 100, and the connecting member 4 side is referred to as the proximal end side (proximal side) A2 of the clip unit 100.

The clip (clip arm) 2 includes a pair of arms 21 that can be opened and closed toward a distal side A1, and a support portion 28 that rotatably supports the pair of arms 21. The support portion 28 is attached to the distal end of the connecting member 4.

The pair of arms 21 includes a first arm 211 and a second arm 212. The first arm 211 and the second arm 212 are arranged on both sides of the central axis O1 in the longitudinal direction A of the clip unit 100. The position of the pair of arms 21 where the first arm 211 and the second arm 212 are arranged symmetrically with respect to the central axis O1 is defined as the “initial position” of the pair of arms 21. Note that the clip 2 may include three or more arms.

The first arm 211 and the second arm 212 are plate-shaped, and the plate thickness direction of the first arm 211 and the second arm 212 substantially coincides with the opening/closing direction P of the first arm 211 and the second arm 212.

The first arm 211 and the second arm 212 include a tissue-grasping portion 22, a flat plate-like grasping portion 23, and a connecting portion 25 connected to the support portion 28 from the distal side A1 toward the proximal side A2. The tissue-grasping portion 22 is formed by bending the distal ends of the first arm 211 and the second arm 212 inward. At the distal ends of the first arm 211 and the second arm 212, claws 24 facing inward are formed.

FIG. 3 is a perspective view of the clip unit 100 with the pair of arms 21 in the swinging state.

The support portion 28 supports the pair of arms 21 so as to be rotatable and swingable (rotatable) about a rotation axis RO extending substantially perpendicular to the longitudinal direction A. A swing direction (rotational direction) Q of the pair of arms 21 about the rotation axis RO substantially coincides with the opening/closing direction P of the pair of arms 21. That is, the plane on which the pair of arms 21 swing (the plane through which the pair of swinging arms 21 pass) substantially coincides with the plane on which the pair of arms 21 open and close (the plane through which the pair of arms 21 that open and close pass). Note that the rotation axis RO is not limited to an axis extending in a direction substantially perpendicular to the longitudinal direction A, and may be an axis extending in a direction intersecting the longitudinal direction A.

When a predetermined force or more is applied to at least one of the first arm 211 and the second arm 212, that is, when at least one of the first arm 211 and the second arm 212 comes into contact with living tissue, or when at least one of the first arm 211 and the second arm 212 is subjected to an external force greater than or equal to a predetermined amount, the support portion 28 swings the pair of arms 21. Otherwise, the support portion 28 does not allow the pair of arms 21 to swing.

The holding tube (tubular member) 3 is a circular tubular member capable of storing at least part of the clip 2. The holding tube 3 has an internal space 38 in which the clip 2 advances and retracts in the longitudinal direction A. The holding tube 3 can fix the clip 2 pulled into the internal space 38 in a closed state.

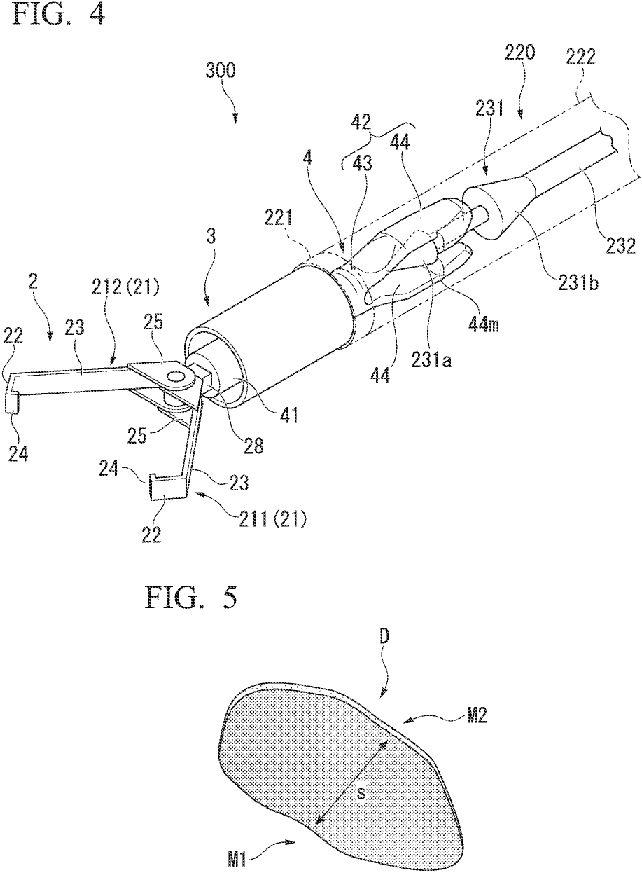

FIG. 4 is a perspective view of the clip unit 100 loaded in the applicator 200.

The connecting member 4 includes an insertion portion 41 inserted into the internal space 38 of the holding tube 3 and a connecting portion 42 provided at the proximal end of the insertion portion 41. The insertion portion 41 is attached to the support portion 28 of the clip 2. The connecting portion 42 is detachably connected to an arrowhead hook portion 231 inserted through the sheath 220.

The insertion portion 41 is formed in a substantially cylindrical rod shape. The insertion portion 41 includes a breaking portion that breaks or deforms when a predetermined amount of breaking force is applied. The pair of arms 21 and the connecting portion 42 are separated by breaking or deforming the breaking portion.

The connecting portion 42 is an engaging portion with which the arrowhead hook portion 231 of the applicator 200 is engaged (connected). The connecting portion 42 includes a connecting portion main body 43 and an elastic arm portion 44.

The elastic arm portion 44 is provided at the proximal end of the connecting portion main body 43 and is bifurcated. The elastic arm portion 44 is elastically deformable with respect to the connecting portion main body 43 and can be opened and closed with respect to the connecting portion main body 43. The elastic arm portion 44 is formed with a notch portion 44m for grasping and housing the engaging portion 231a of the arrowhead hook portion 231. The notch portion 44m is formed in a shape that closely contacts the outer peripheral surface of the engaging portion 231a of the arrowhead hook portion 231.

### Suture Treatment Using Clip Unit 100

Next, suture treatment using the clip unit 100 will be described. Specifically, a treatment for suturing a defective portion D formed in a body tissue in the body as shown in FIG. 5 by endoscopic treatment will be described.

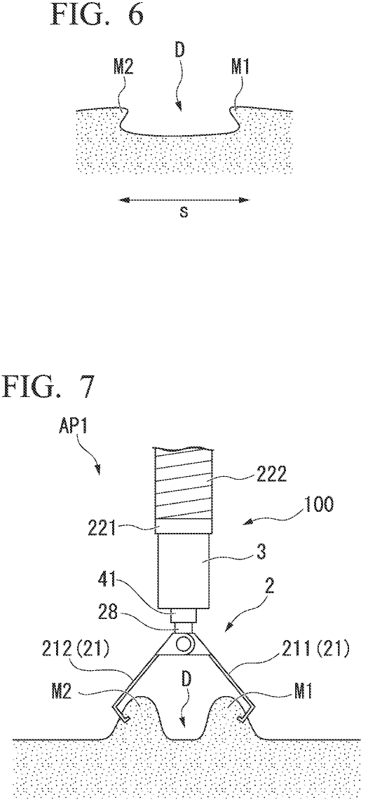

FIG. 6 is a cross-sectional view of the defective portion D in the sewing direction S.

The operator brings the endoscope closer to the defect D. The operator observes the defect D and determines the suturing direction S in which the defect D is sutured based on the size and shape of the defect D. In the following description, the mucous membranes surrounding the defective portion D, which are arranged facing each other in the suturing direction S across the defective portion D. are referred to as “first mucous membrane M1” and “second mucous membrane M2”.

### Approach Step

The operator introduces the clip unit 100 loaded into the applicator 200 into the body through the channel of the endoscope. The operator moves the endoscope and the sheath 220 to bring the clip 2 of the clip unit 100 closer to the defect D, which is the treatment area.

There are at least a front approach AP1 and a tangential approach AP2 as methods for approaching the treatment area with the clip unit 100. In the front approach AP1, the operator operates the endoscope and the sheath 220 to position the clip unit 100 so that the longitudinal direction A of the clip unit 100 is substantially perpendicular to the surface of the treatment area. In the tangential approach AP2. the operator operates the endoscope and the sheath 220 to arrange the clip unit 100 so that the longitudinal direction A of the clip unit 100 is along the surface of the treatment area.

### Front Approach AP1

FIG. 7 is a diagram showing the clip unit 100 for treating the defect D by the front approach AP1. The operator brings the clip unit 100 closer to the defect D, which is the treatment area, by a front approach AP1. The pair of arms 21 are arranged at the initial position. A pair of arms 21 are arranged to face the front of the defect D. which is the treatment area. The first mucous membrane M1 and the second mucous membrane M2 are grasped by a pair of arms 21.

### Tangential Approach AP2

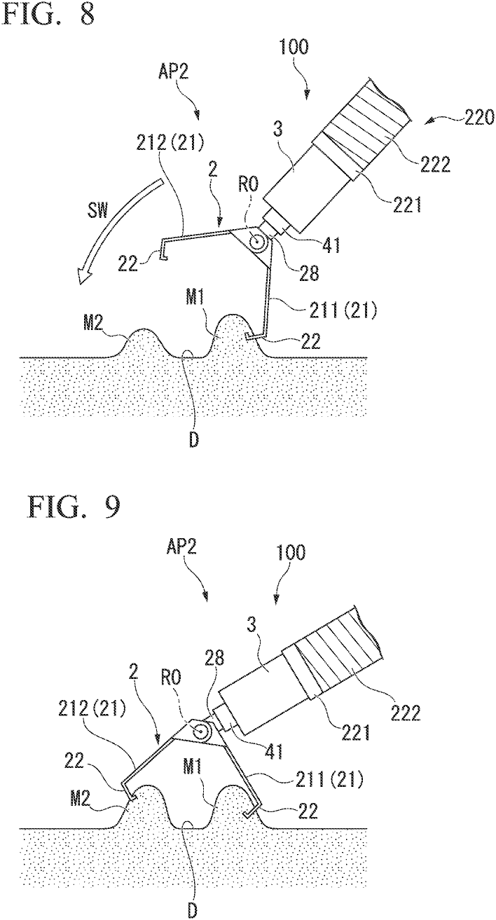

FIG. 8 is a diagram showing the clip unit 100 used to treat the defect D by the tangential approach AP2. The operator brings the clip unit 100 closer to the defect D, which is the treatment area, by the tangential approach AP2. The pair of arms 21 are arranged at the initial position. The operator hooks the tissue-grasping portion 22 of the first arm 211 on the mucous membrane (the first mucous membrane M1 in FIG. 8) on the endoscope side (front side) of the first mucous membrane M1 and the second mucous membrane M2.

FIG. 9 is a diagram showing the clip unit 100 with the pair of arms 21 in the swinging state. The operator brings the clip unit 100 with the tissue-grasping portion 22 hooked on the first mucous membrane M1 closer to the defect D, which is the treatment area. Specifically, the operator advances the applicator 200 with respect to the treatment instrument channel of the endoscope. Alternatively, the operator brings the clip unit 100 closer to the defect D by advancing the endoscope . As a result, as shown in FIG. 9, the pair of arms 21 swing (SW) around the rotation axis RO, and the second arm 212 comes into contact with the second mucous membrane M2. Specifically, the support portion 28 swings the first arm 211 in a direction to approach the sheath 220. The support portion 28 swings the second arm 212 away from the sheath 220. As a result, the pair of arms 21 are arranged to face the front of the defect D, which is the treatment area. The first mucous membrane M1 and the second mucous membrane M2 are grasped by the pair of arms 21.

### Towing Step

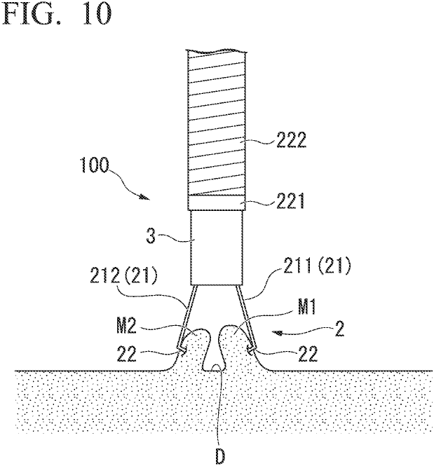

FIG. 10 is a diagram showing a pulling step using the clip unit 100.

The operator retracts the arrowhead hook portion 231 by retracting the slider 242 along the operation portion main body 241. The connecting member 4 connected to the arrowhead hook portion 231 pulls the clip 2. The clip 2 pulled by the connecting member 4 is drawn into the holding tube 3. When the clip 2 is pulled into the holding tube 3 to a predetermined position, the clip 2 is restricted from moving toward the distal side A1 with respect to the holding tube 3, and the pair of arms 21 are locked in the closed state. Once the pair of arms 21 are locked in the closed state, the pair of arms 21 cannot return to the open state.

Note that the applicator 200 shown in FIG. 10 is arranged so that the applicator 200 is substantially perpendicular to the surface of the treatment area by the front approach AP1. When the applicator 200 is placed by the tangential approach AP2, the applicator 200 is placed along the surface of the treatment area.

### Separation Step

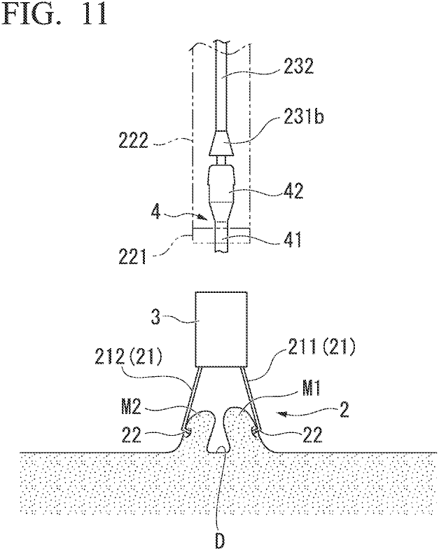

FIG. 11 is a diagram showing the separation step using the clip unit 100.

The operator further pulls the clip 2. A breaking force amount is applied to the breaking portion of the insertion portion 41 of the connecting member 4 by pulling, and the breaking portion breaks. As a result, the pair of arms 21 and the connecting portion 42 are separated. The operator withdraws the sheath 220 and leaves the clip 2 and the holding tube 3 in a state of ligating the first mucous membrane M1 and the second mucous membrane M2 in the body.

According to the clip unit 100 of the present embodiment, the treatment of suturing the defect D can be preferably performed. Even when the defect D is treated by the tangential approach AP2, the pair of arms 21 are swung about the rotation axis RO so that the pair of arms 21 can be arranged to face the front of the defect D. which is the treatment area.

The first embodiment of the present invention has been described in detail above with reference to the drawings, but the specific configuration is not limited to this embodiment, and design changes and the like are included within the scope of the present invention. Also, the constituent elements shown in the above-described embodiment and modifications shown below can be combined as appropriate.

### Modification 1-1

FIG. 12 is a diagram showing a sheath 220A that is a modification of the sheath 220 of the applicator 200. The sheath 220A includes a distal tip 221, a distal coil 222, a proximal coil 224, and a restriction portion 225. The restriction portion 225 is provided along the longitudinal direction A on the outer peripheral surface of the distal coil 222. The restriction portion 225 is provided only in a part of the circumferential direction C with respect to the longitudinal direction A. The restriction portion 225 is a portion that fixes the outer peripheral surface of the distal coil 222. The restriction portion 225 is, for example, a portion where the outer peripheral surface of the distal coil 222 is welded. The restriction portion 225 may be a portion in which a plate member is joined to the outer peripheral surface of the distal coil 222 by welding or the like.

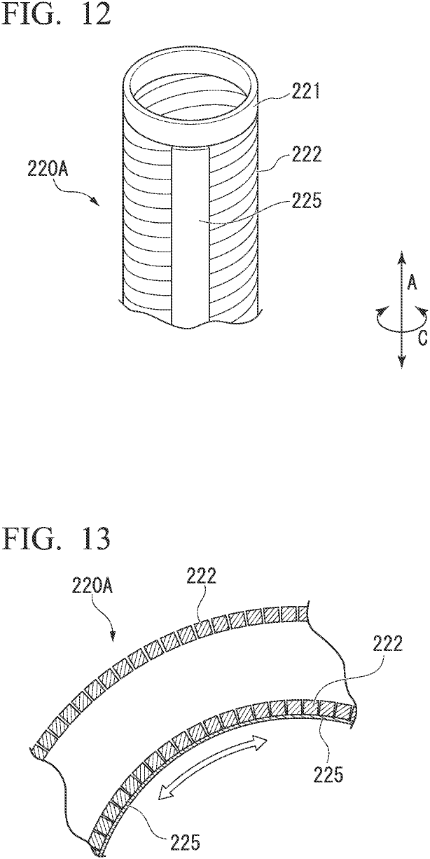

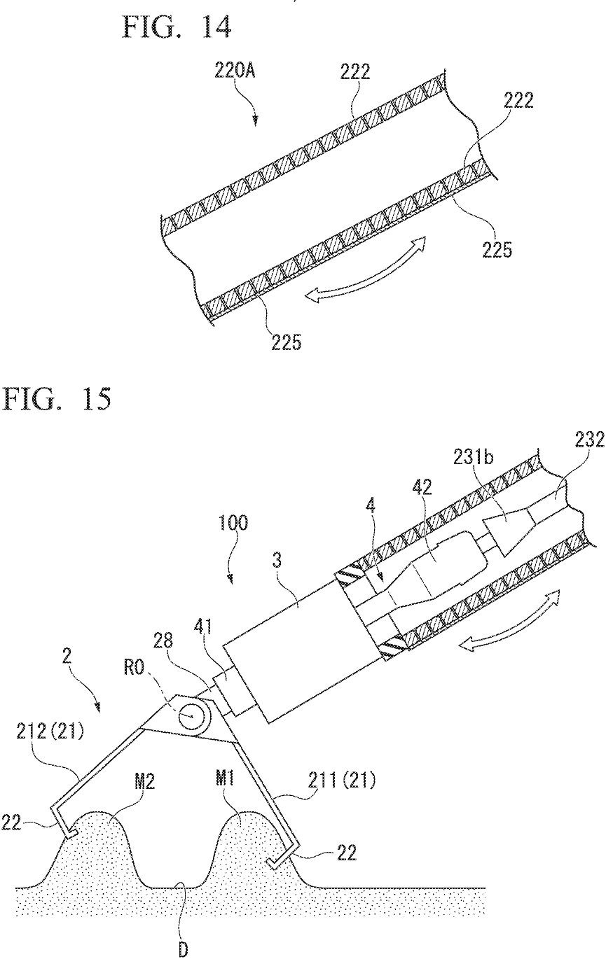

FIGS. 13 and 14 are diagrams showing the operation of the sheath 220A.

If the distal coil 222 of the sheath 220A is bent to the side where the restriction portion 225 is provided with respect to the central axis of the longitudinal direction A of the sheath 220A, the distal coil 222 of the sheath 220A flexes similarly to the distal coil 222 of the sheath 220, as shown in FIG. 13. On the other hand, if the distal side coil 222 is bent to the side opposite to the side on which the restriction portion 225 is provided with respect to the central axis of the sheath 220A in the longitudinal direction A, the restriction portion 225 fixes the outer peripheral surface of the distal side coil 222, so that the distal coil 222 of the sheath 220A does not flex as shown in FIG. 14.

FIG. 15 is a diagram showing an approach step using the sheath 220A.

When the operator grasps the first mucous membrane M1 and the second mucous membrane M2 with the pair of arms 21 by the tangential approach AP2. it is necessary to press the pair of arms 21 against the living tissue. At this time, if the regulating portion 225 of the sheath 220A is arranged in the direction in which the pair of arms 21 swing with respect to the central axis of the longitudinal direction A of the sheath 220A (living tissue side), the pair of arms 21 can be moved to the living tissue. When pressed against the distal side coil 222. it does not bend and is easy to press . The sheath 220A may include a marker that facilitates visual recognition of the position where the restriction portion 225 is provided with an endoscope.

### Second Embodiment

A clip system 300B according to a second embodiment of the present invention will be described with reference to FIGS. 16 to 20. In the following description, the same reference numerals are given to the same configurations as those already described, and redundant descriptions will be omitted.

The clip system 300B includes a clip unit 100B and an applicator 200. The clip unit 100B is loaded into the applicator 200 that can be inserted through a channel of an endoscope and introduced to a treatment position.

FIG. 16 is a perspective view of the clip unit 100B according to this embodiment.

The clip unit 100B includes a clip 2B, a holding tube 3, and a connecting member 4.

The clip (clip arm) 2B includes a pair of arms 21B that can be opened and closed toward the distal side A1, and a support portion 28 that swingably supports the pair of arms 21B. Note that the support portion 28 may support the pair of arms 21B to prevent swinging.

The pair of arms 21B includes a first arm 211B and a second arm 212B. The first arm 211B and the second arm 212B are arranged on both sides of the central axis O1 in the longitudinal direction A.

The first arm 211B and the second arm 212B include a tissue-grasping portion 22B. a flat plate-like grasping portion 23, and a connecting portion 25 from the distal side A1 to the proximal end side A2. The tissue-grasping portion 22B is formed by bending the distal ends of the first arm 211B and the second arm 212B inward.

The tissue-grasping portion 22B includes a distal end portion (anchor) 26a provided at the distal end of the tissue-grasping portion 22B. The distal end portion (anchor) 26a is formed in a substantially disc shape, and the plate thickness direction substantially coincides with the opening/closing direction P of the pair of arms 21B. In the vertical direction B substantially perpendicular to the longitudinal direction A and the opening/closing direction P, the length of the distal end portion (anchor) 26a is longer than the length of the other portion of the tissue-grasping portion 22B.

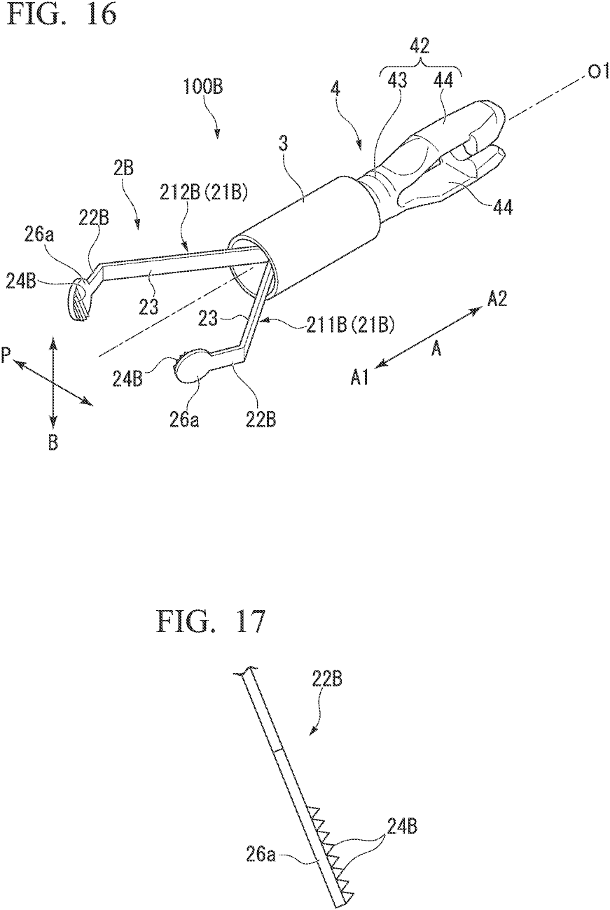

FIG. 17 is a side view of the tissue-grasping portion 22B.

The distal end portion (anchor) 26a of the tissue-grasping portion 22B includes a claw 24B inside in the opening/closing direction P. The claw 24B extends inward in the opening/closing direction P.

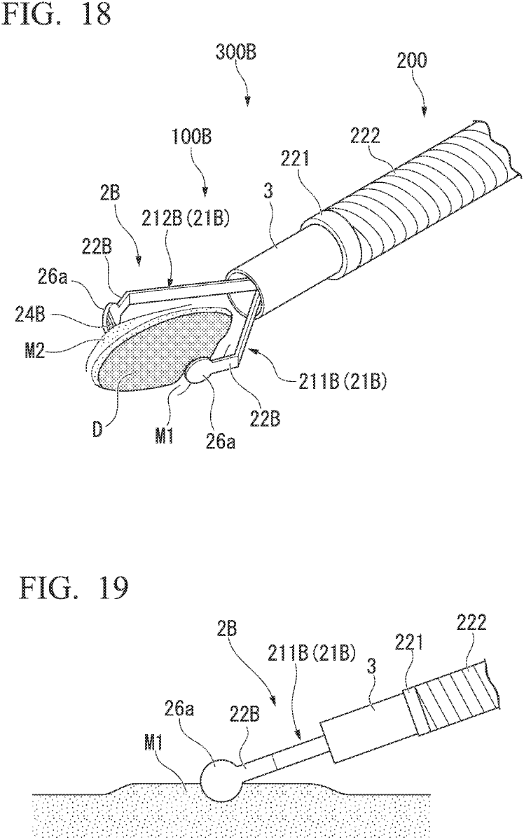

FIG. 18 is a perspective view of a pair of arms 21B that grasp the first mucous membrane M1 and the second mucous membrane M2. FIG. 19 is a side view of a pair of arms 21B that grasp the first mucous membrane M1 and the second mucous membrane M2. The operator approaches the clip unit 100B with the tangential approach AP2 to the defect D, which is the treatment area. The operator can securely grasp the first mucous membrane M1 and the second mucous membrane M2 with the distal end portion (anchor) 26a having the claw 24B.

FIG. 20 is a diagram showing a pulling step using the clip unit 100B.

The operator pulls the clip 2 to a predetermined position on the holding tube 3 to lock the pair of arms 21B in the closed state. Since the distal end portion (anchor) 26a having the claw 24B securely grasps the first mucous membrane M1 and the second mucous membrane M2, the distal end portion (anchor) 26a is less likely to slip from the first mucous membrane M1 and the second mucous membrane M2.

According to the clip unit 100B of the present embodiment, the treatment of suturing the defect D can be preferably performed. The pair of arms 21B can reliably grasp the first mucous membrane M1 and the second mucous membrane M2.

The present invention has been described in detail above with reference to the drawings, but the specific configuration is not limited to this embodiment, and design changes and the like are also included within the scope of the present invention. Also, the constituent elements shown in the above-described embodiment and modifications shown below can be combined as appropriate.

### Third Embodiment

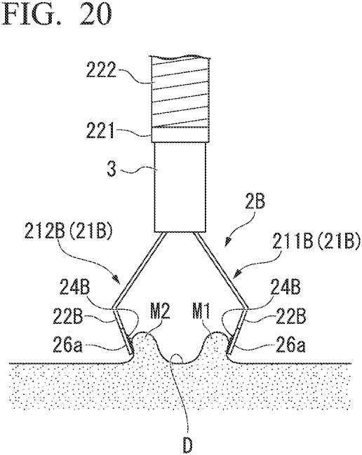

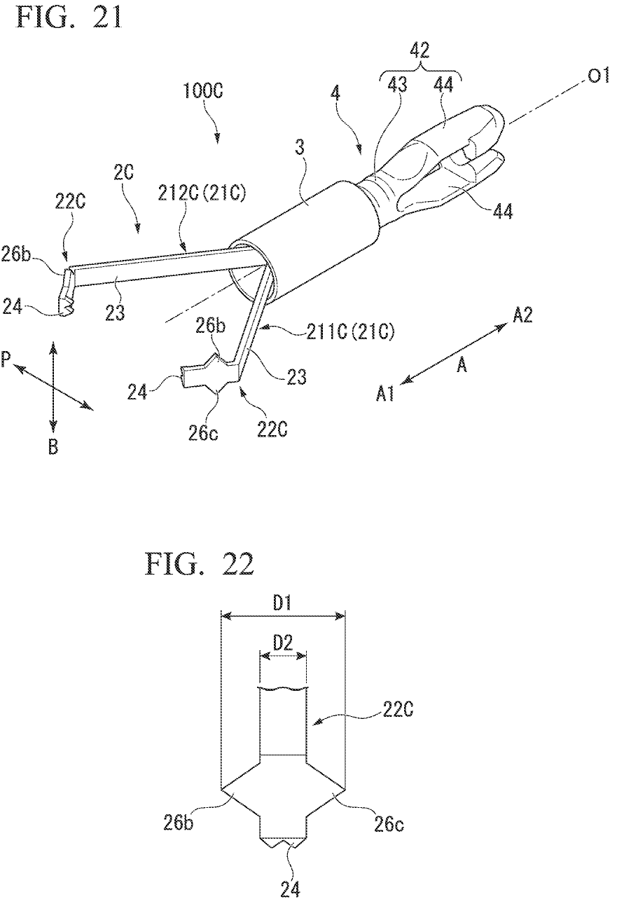

A clip system 300C according to a third embodiment of the present invention will be described with reference to FIGS. 21 to 23. In the following description, the same reference numerals are given to the same configurations as those already described, and redundant descriptions will be omitted.

The clip system 300C includes a clip unit 100C and an applicator 200. The clip unit 100C is loaded into an applicator 200 that can be inserted through a channel of an endoscope and introduced to a treatment position.

FIG. 21 is a perspective view of a clip unit 100C according to this embodiment.

The clip unit 100C includes a clip 2C, a holding tube 3, and a connecting member 4.

The clip (clip arm) 2C includes a pair of arms 21C that can be opened and closed toward the distal side A1, and a support portion 28 that swingably supports the pair of arms 21C. Note that the support portion 28 may support the pair of arms 21C to prevent swinging.

The pair of arms 21C includes a first arm 211C and a second arm 212C. The first arm 211C and the second arm 212C are arranged on both sides of the central axis O1 in the longitudinal direction A.

The first arm 211C and the second arm 212C include a tissue-grasping portion 22C, a flat plate-like grasping portion 23, and a connecting portion 25 from the distal side A1 to the proximal end side A2. The tissue-grasping portion 22C is formed by bending the distal ends of the first arm 211B and the second arm 212B inward.

FIG. 22 is a side view of the tissue-grasping portion 22C.

The tissue-grasping portion 22C includes a first anchor 26b and a second anchor 26c. The first anchor 26b and the second anchor 26c are provided on both sides in the vertical direction B of the tissue-grasping portion 22C. The first anchor 26b and the second anchor 26c are formed in a sharp shape protruding in the vertical direction B. In the vertical direction B, the length D1 from the distal end of the first anchor 26b to the distal end of the second anchor 26c is longer than the length D2 of the other portion of the tissue-grasping portion 22B.

FIG. 23 is a perspective view of a pair of arms 21C that grasp the first mucous membrane M1 and the second mucous membrane M2. The operator brings the clip unit 100C closer to the defect D. which is the treatment area, by the tangential approach AP2. Among the first anchor 26b and the second anchor 26c, the anchor on the biological tissue side (the second anchor 26c in FIG. 23) penetrates the biological tissue. The operator can reliably grasp the first mucous membrane M1 and the second mucous membrane M2 by the tissue-grasping portion 22C having the first anchor 26b and the second anchor 26c.

According to the clip unit 100C of the present embodiment, the treatment of suturing the defect D can be preferably performed. The pair of arms 21C can reliably grasp the first mucous membrane M1 and the second mucous membrane M2.

The third embodiment of the present invention has been described in detail above with reference to the drawings, but the specific configuration is not limited to this embodiment, and design changes and the like are included within the scope of the present invention. Also, the constituent elements shown in the above-described embodiment and modifications shown below can be combined as appropriate.

### Fourth Embodiment

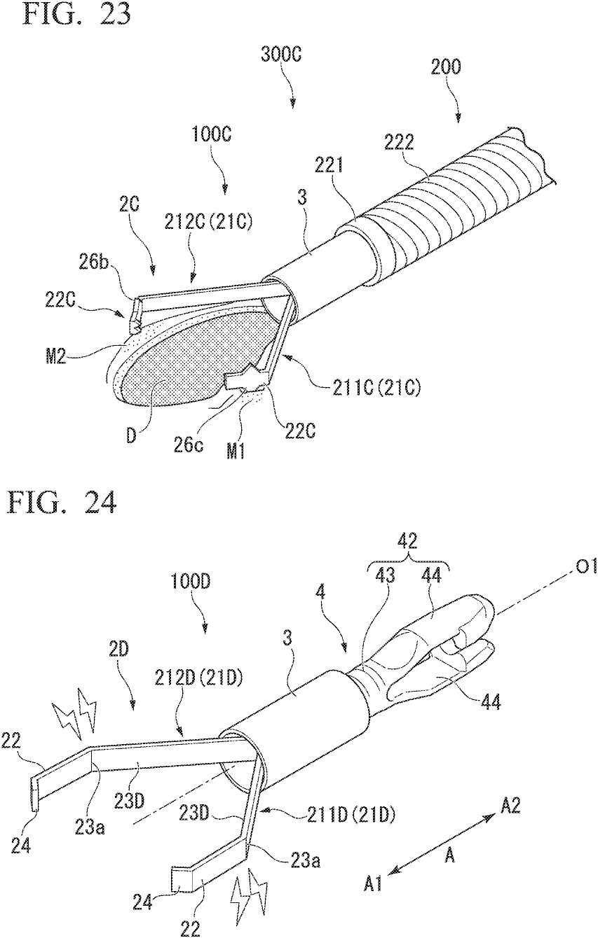

A clip system 300D according to the fourth embodiment of the present invention will be described with reference to FIGS. 24 to 29. In the following description, the same reference numerals are given to the same configurations as those already described, and redundant descriptions will be omitted.

The clip system 300D includes a clip unit 100D and an applicator 200D. The clip unit 100D is loaded into an applicator 200D that can be inserted through a channel of an endoscope and introduced to a treatment position.

FIG. 24 is a perspective view of a clip unit 100D according to this embodiment.

The clip unit 100D includes a clip 2D, a holding tube 3, and a connecting member 4. The clip unit 100D is used by being loaded on the applicator 200D, and can supply high-frequency current supplied from the arrowhead hook portion 231 connected to the connecting member 4 to the clip 2D.

The clip 2D includes a pair of arms 21D that can be opened and closed toward the distal side A1, and a support portion 28 that swingably supports the pair of arms 21D. Note that the support portion 28 may support the pair of arms 21D to prevent swinging.

The pair of arms 21D includes a first arm 211D and a second arm 212D. The first arm 211D and the second arm 212D are arranged on both sides of the central axis O1 in the longitudinal direction A. At least one of the first arm 211D and the second arm 212D has conductivity.

The first arm 211D and the second arm 212D include a tissue-grasping portion 22, a grasping portion 23D. and a connecting portion 25 from the distal side A1 toward the proximal side A2. The grasping portion 23D includes a bent portion 23a in which the distal side A1 is bent inward.

FIG. 25 is a diagram showing an applicator 200D loaded with a clip unit 100D. The applicator 200D includes a sheath 220, an operation wire 230, and an operation portion 240D.

The operation portion 240D includes an operation portion main body 241, a slider 242D, a connector 247, and a thumb ring 248.

The slider 242D is attached movably back and forth in the longitudinal axis direction of the operation portion main body 241, and the proximal end of the wire 232 is attached to the slider 242D. As the slider 242D advances and retracts along the operation portion main body 241, the wire 232 advances and retracts with respect to the sheath 220. and the arrowhead hook portion 231 advances and retracts.

The connector 247 can be connected to a high-frequency power supply (not shown), and is electrically and physically connected to the proximal end of the wire 232. The connector 247 can supply the high frequency current supplied from the high frequency power supply to the connecting member 4 and the clip 2D via the wire 232. The connector 247 is supported by the slider 242D and has a substantially cylindrical shape.

### Suture Treatment Using Clip Unit 100D

Next, suture treatment using the clip unit 100D will be described. Specifically, a treatment for suturing a defective portion D formed in a body tissue in the body as shown in FIG. 5 by endoscopic treatment will be described.

### Approach Step

The operator introduces the clip unit 100D loaded into the applicator 200D into the body through the channel of the endoscope. The operator moves the endoscope and the sheath 220 to bring the clip 2D of the clip unit 100D closer to the defect D. which is the treatment area. The operator advances and retracts the wire 232 to deploy the clip 2D in an appropriate state.

### First Grasping Step

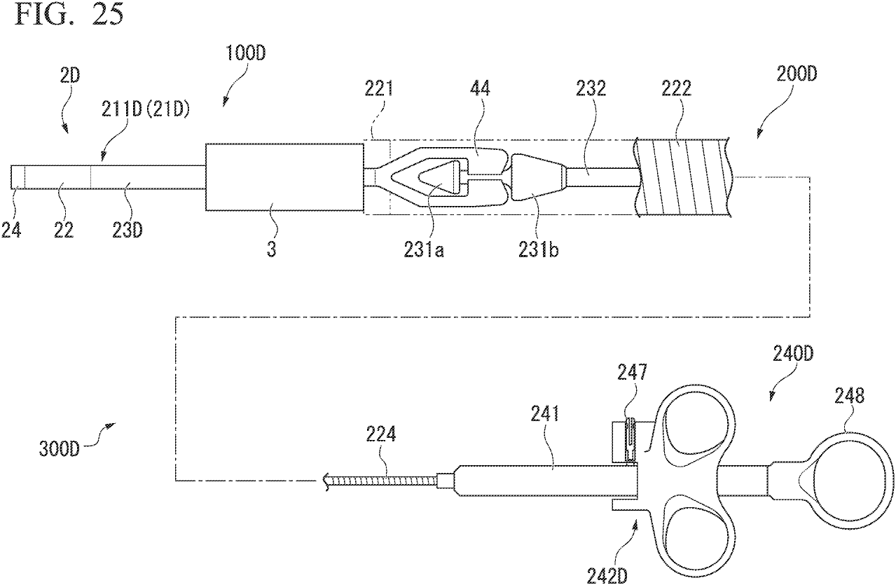

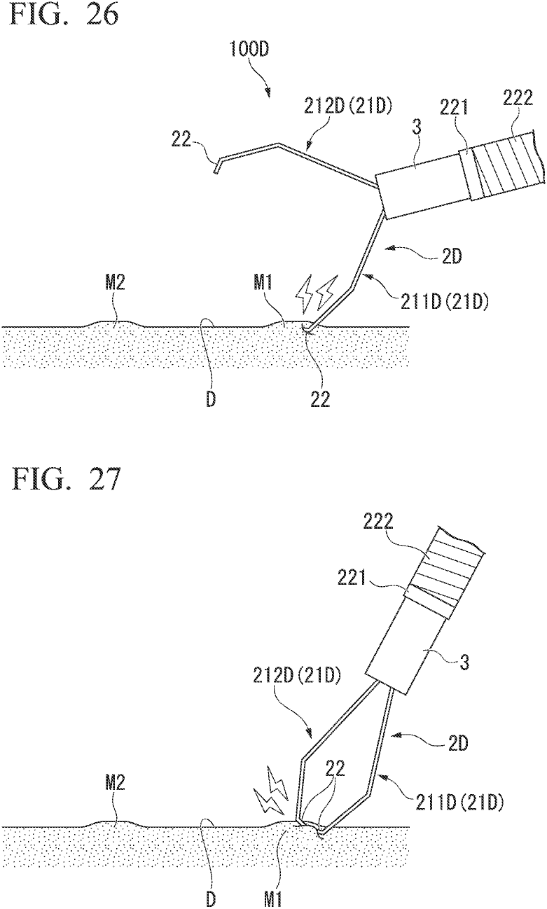

FIGS. 26 and 27 are diagrams showing the first grasping step using the clip unit 100D. The operator makes a small hole in the edge of the defect D by applying a high-frequency current to the clip 2D. Alternatively, the operator damages the living tissue to form a bump (hook). For example, as shown in FIG. 26, the operator applies high-frequency current to the first arm 211D to make a hole in the first mucous membrane M1. The operator may pass the high-frequency current while holding the first mucous membrane M1 with the first arm 211D and the second arm 212D as shown in FIG. 26. The operator grasps the first mucous membrane M1 by inserting the distal end of the first arm 211D into the formed small hole or bump (hook). Since the distal end of the first arm 211D is inserted into the formed small hole or bump (hook), it does not slip.

### Second Grasping Step

FIG. 28 is a diagram showing the second grasping step using the clip unit 100D.

The operator moves the clip unit 100D along the suturing direction S to move the distal end of the second arm 212D to the vicinity of the second mucous membrane M2. The operator grasps the second mucous membrane M2 with the second arm 212D in the same manner as the method of grasping the first mucous membrane M1 with the first arm 21D.

### Hemostasis Step

When bleeding occurs from the living tissue, the operator may apply a high-frequency current to the clip 2D to cauterize the bleeding site and perform hemostatic treatment.

### Towing Step

The operator retracts the arrowhead hook portion 231 by retracting the slider 242D along the operation portion main body 241. The connecting member 4 connected to the arrowhead hook portion 231 pulls the clip 2D. The clip 2D pulled by the connecting member 4 is drawn into the holding tube 3. When the clip 2D is pulled to a predetermined position on the holding tube 3, the clip 2D is restricted from moving toward the distal side with respect to the holding tube 3, and the pair of arms 21D are locked in the closed state. Once the pair of arms 21D are locked in the closed state, the pair of arms 21D cannot return to the open state.

### Separation Step

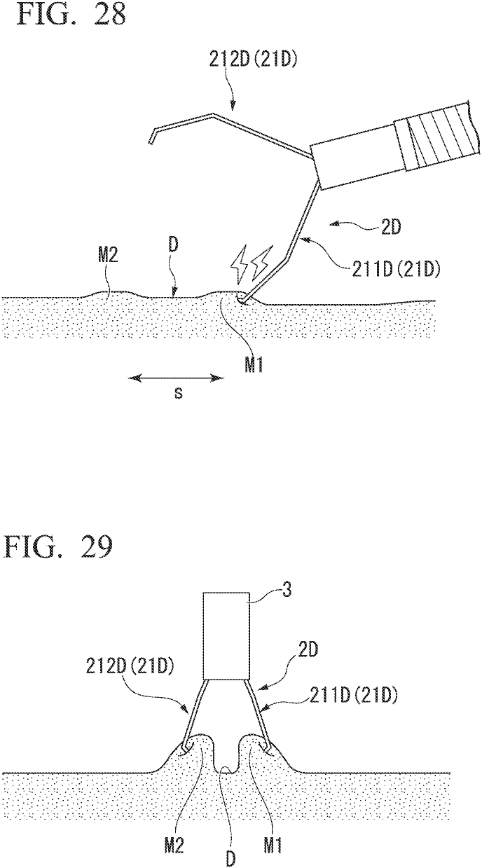

FIG. 29 is a diagram showing the separation step using the clip unit 100D.

The operator further pulls Clip 2D. A breaking force amount is applied to the breaking portion of the insertion portion 41 of the connecting member 4 by pulling, and the breaking portion breaks. As a result, the pair of arms 21D and the connecting portion 42 are separated. The operator withdraws the sheath 220 and leaves the clip 2D and the holding tube 3 in a state of ligating the first mucous membrane M1 and the second mucous membrane M2 in the body.

According to the clip unit 100D of the present embodiment, the treatment of suturing the defect D can be preferably performed. By inserting the distal end of the clip 2D into a small hole or bump (hook) formed by the high-frequency current, the clip 2D does not easily slip on the tissue.

The fourth embodiment of the present invention has been described above in detail with reference to the drawings, but the specific configuration is not limited to this embodiment, and design changes and the like are included within the scope of the present invention. Also, the constituent elements shown in the above-described embodiment and modifications shown below can be combined as appropriate.

### Fifth Embodiment

A clip system 300E according to a fifth embodiment of the present invention will be described with reference to FIGS. 30 to 34. In the following description, the same reference numerals are given to the same configurations as those already described, and redundant descriptions will be omitted.

The clip system 300E includes a clip unit 100E and an applicator 200D. The clip unit 100E is loaded into an applicator 200D that can be inserted through a channel of an endoscope and introduced to a treatment position.

FIG. 30 is a perspective view of a clip unit 100E according to this embodiment.

The clip unit 100E includes a clip 2E, a holding tube 3, and a connecting member 4. The clip unit 100E is used by being loaded into the applicator 200D, and can supply high-frequency current supplied from the arrowhead hook portion 231 connected to the connecting member 4 to the clip 2E.

The clip 2E includes a pair of arms 21E that can be opened and closed toward the distal side A1, and a support portion 28 that swingably supports the pair of arms 21E. Note that the support portion 28 may support the pair of arms 21E to prevent swinging.

The pair of arms 21E includes a first arm 211E and a second arm 212E. The first arm 211E and the second arm 212E are arranged on both sides of the central axis O1 in the longitudinal direction A. At least one of the first arm 211E and the second arm 212E has conductivity.

The first arm 211E and the second arm 212E include a tissue-grasping portion 22, a grasping portion 23, and a connecting portion 25 from the distal side A1 toward the proximal side A2.

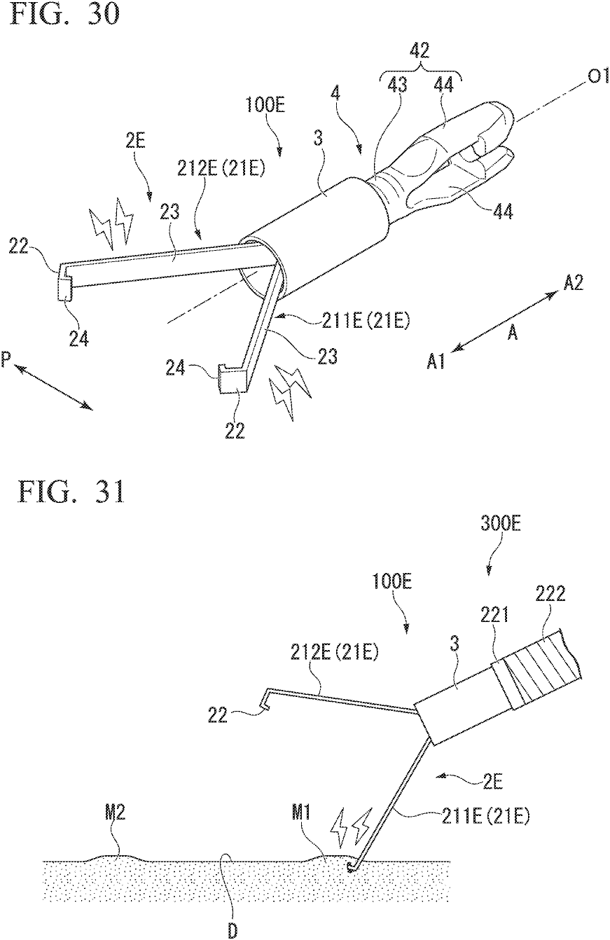

FIG. 31 is a diagram showing the first grasping step using the clip unit 100E.

The operator applies high-frequency current to the first arm 211E to make a hole in the first mucous membrane M1. The operator grasps the first mucous membrane M1 by inserting the distal end of the first arm 211E into the formed small hole. Since the distal end of the first arm 211E is inserted into the formed small hole, it does not slip.

FIG. 32 is a diagram showing the second grasping step using the clip unit 100E.

The operator moves the clip unit 100E along the suturing direction S to move the distal end of the second arm 212D to the vicinity of the second mucous membrane M2. The operator grasps the second mucous membrane M2 with the second arm 212D in the same manner as the method of grasping the first mucous membrane M1 with the first arm 211E.

### Hemostasis Step

When bleeding occurs from the living tissue, the operator may apply a high-frequency current to the clip 2E to cauterize the bleeding site and perform hemostatic treatment.

### Towing Step

The operator retracts the arrowhead hook portion 231 by retracting the slider 242 along the operation portion main body 241. The connecting member 4 connected to the arrowhead hook portion 231 pulls the clip 2E. The clip 2E pulled by the connecting member 4 is drawn into the holding tube 3. When the clip 2E is pulled to a predetermined position on the holding tube 3, the clip 2E is restricted from moving toward the distal side with respect to the holding tube 3, and the pair of arms 21E are locked in the closed state. Once the pair of arms 21E are locked in the closed state, the pair of arms 21E cannot return to the open state.

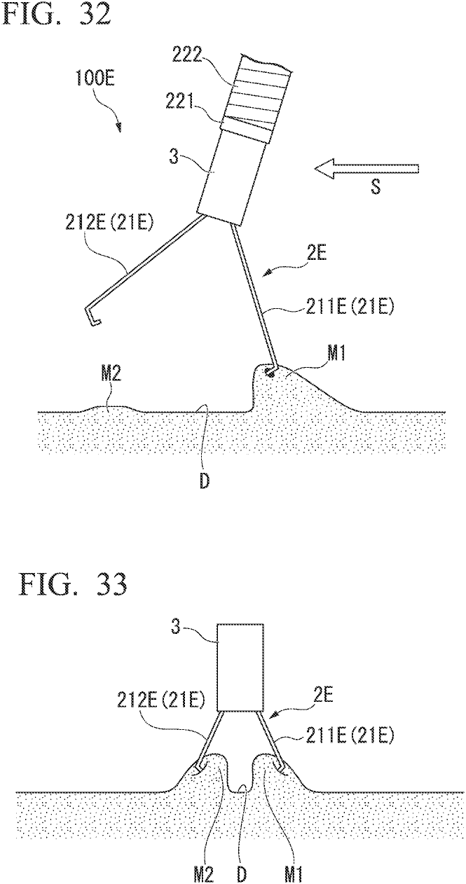

FIG. 33 is a diagram showing the separation step using the clip unit 100E.

The operator further pulls the clip 2E. A breaking force amount is applied to the breaking portion of the insertion portion 41 of the connecting member 4 by pulling, and the breaking portion breaks. As a result, the pair of arms 21E and the connecting portion 42 are separated. The operator withdraws the sheath 220 and leaves the clip 2E and the holding tube 3 in a state of ligating the first mucous membrane M1 and the second mucous membrane M2 in the body.

FIG. 34 is a diagram showing another aspect of the first grasping step and the second grasping step using the clip unit 100E. The operator may supply high-frequency current to the clip 2E while both the first arm 211E and the second arm 212E are in contact with the living tissue.

According to the clip unit 100E of the present embodiment, the treatment of suturing the defect D can be preferably performed. By inserting the distal end of the clip 2E into the small hole formed by the high-frequency current, the clip 2E does not easily slip on the tissue.

The fifth embodiment of the present invention has been described in detail above with reference to the drawings, but the specific configuration is not limited to this embodiment, and design changes and the like are included within the scope of the present invention. Also, the constituent elements shown in the above-described embodiment and modifications shown below can be combined as appropriate.

### Sixth Embodiment

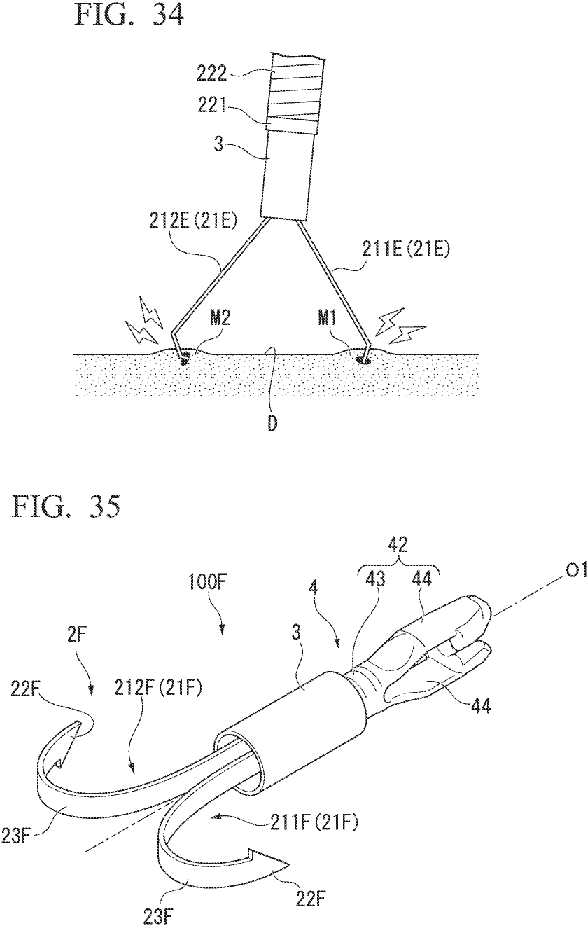

A clip system 300F according to the sixth embodiment of the present invention will be described with reference to FIGS. 35 to 39. In the following description, the same reference numerals are given to the same configurations as those already described, and redundant descriptions will be omitted.

The clip system 300F includes a clip unit 100F and an applicator 200. The clip unit 100F is loaded into an applicator 200 that can be inserted through a channel of an endoscope and introduced to a treatment position.

FIG. 35 is a perspective view of the clip unit 100F according to this embodiment.

The clip unit 100F includes a clip 2F, a holding tube 3, and a connecting member 4.

The clip 2F includes a pair of arms 21F that can be opened and closed toward the distal side A1, and a support portion 28 that swingably supports the pair of arms 21F. Note that the support portion 28 may support the pair of arms 21F to prevent swinging.

The pair of arms 21F includes a first arm 211F and a second arm 212F. The first arm 211F and the second arm 212F are arranged on both sides of the central axis O1 in the longitudinal direction A.

The first arm 211F and the second arm 212F include a tissue-grasping portion 22F, a grasping portion 23F, and a connecting portion 25 from the distal side A1 to the proximal side A2. The tissue-grasping portion 22F is formed in an arrowhead shape with a sharp distal end. The tissue-grasping portion 22F may include barbs. The grasping portion 23F is formed in a hook-like shape, with the distal end side curved largely in a direction (outward) away from the central axis O1.

FIG. 36 is a diagram showing the first grasping step using the clip unit 100F.

The operator punctures the first mucous membrane M1 with the distal end of the first arm 211F. Specifically, the operator punctures the edge of the defect D from the inside to the outside with the distal end of the first arm 211F.

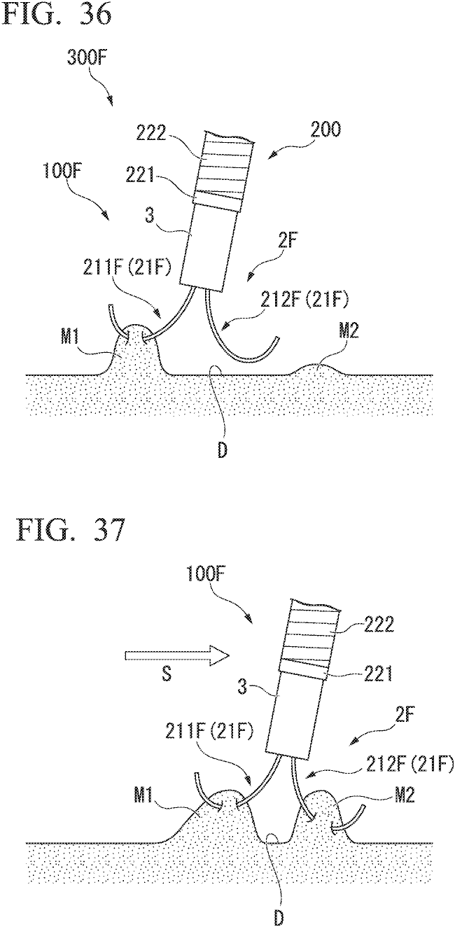

FIG. 37 is a diagram showing the second grasping step using the clip unit 100F.

The operator moves the clip unit 100F along the suturing direction S to move the distal end of the second arm 212F to the vicinity of the second mucous membrane M2. The operator punctures the second mucous membrane M2 with the distal end of the second arm 212F. Specifically, the operator punctures the edge of the defect D from the inside to the outside with the distal end of the second arm 212F.

FIG. 38 is a diagram showing a pulling step using the clip unit 100F.

The operator retracts the arrowhead hook portion 231 by retracting the slider 242 along the operation portion main body 241. The connecting member 4 connected to the arrowhead hook portion 231 pulls the clip 2F. The pair of arms 21F are drawn together to draw the first mucous membrane M1 and the second mucous membrane M2.

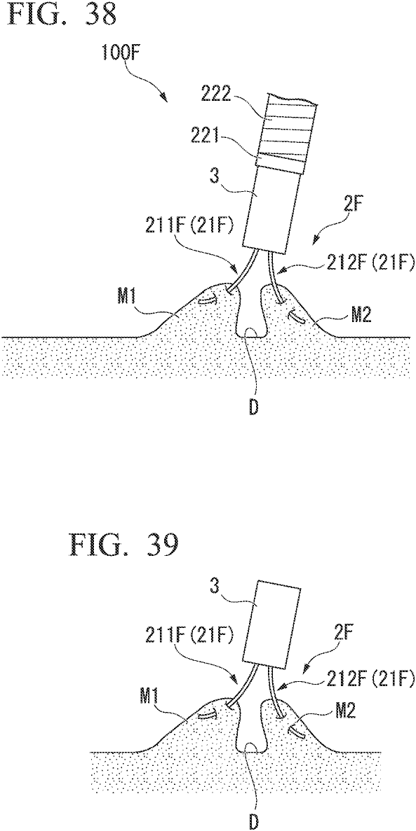

FIG. 39 is a diagram showing the separation step using the clip unit 100F.

The operator withdraws the sheath 220 and leaves the clip 2F and the holding tube 3 in a state of ligating the first mucous membrane M1 and the second mucous membrane M2 in the body.

According to the clip unit 100F of the present embodiment, the treatment of suturing the defect D can be preferably performed. The pair of arms 21F can reliably grasp the first mucous membrane M1 and the second mucous membrane M2.

The sixth embodiment of the present invention has been described in detail above with reference to the drawings, but the specific configuration is not limited to this embodiment, and design changes and the like are included within the scope of the present invention. Also, the constituent elements shown in the above-described embodiment and modifications shown below can be combined as appropriate.

### Seventh Embodiment

A clip system 300G according to the seventh embodiment of the present invention will be described with reference to FIGS. 40 to 45. In the following description, the same reference numerals are given to the same configurations as those already described, and redundant descriptions will be omitted.

The clip system 300G includes a clip unit 100G and an applicator 200. The clip unit 100G is loaded into an applicator 200 that can be inserted through a channel of an endoscope and introduced to a treatment position.

FIG. 40 is a perspective view of a clip unit 100G according to this embodiment.

The clip unit 100G includes a clip 2G, a holding tube 3, and a connecting member 4.

The clip (clip arm) 2G includes a pair of arms 21G that can be opened and closed toward the distal side A1, and a support portion 28 that swingably supports the pair of arms 21G. Note that the support portion 28 may support the pair of arms 21G to prevent swinging.

The pair of arms 21G includes a first arm 211G and a second arm 212. The first arm 211G and the second arm 212 are arranged on both sides of the central axis O1 in the longitudinal direction A.

The first arm 211G includes a tissue-grasping portion 22, a flat plate-like grasping portion 23, a connecting portion 25, and a connecting ring 27. The connecting ring 27 is formed in a ring shape, and is provided outside in the opening/closing direction P at the distal end portion of the grasping portion 23. A second arm 212 of another clip unit 100G can be inserted through the connecting ring 27. The connecting ring 27 may be provided at a portion where the first arm 211G curves. In addition, the second arm 212 may include the connecting ring 27.

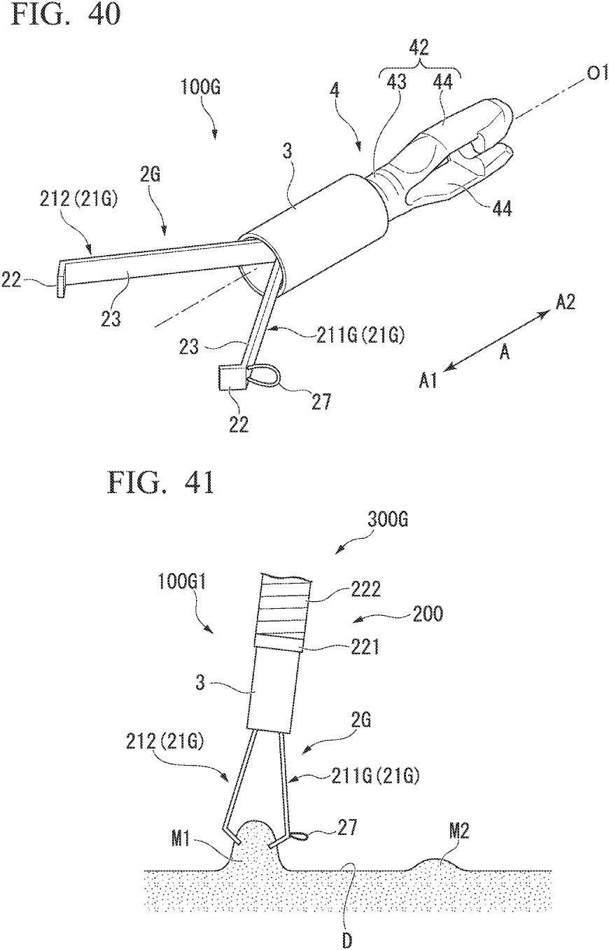

FIG. 41 is a diagram showing the first grasping step using the clip unit 100G.

The operator grasps the first mucous membrane M1 by ligating the pair of arms 21G of the clip 2G of the first clip unit 100G (hereinafter also referred to as “clip unit 100G1”).

FIG. 42 is a diagram showing the first separation step using the clip unit 100G.

The operator withdraws the sheath 220 and leaves the clip 2G and the holding tube 3 in a state of ligating only the first mucous membrane M1.

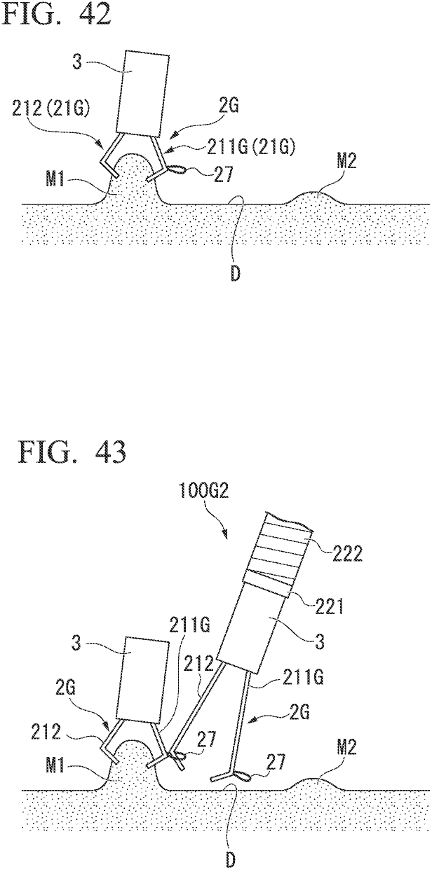

FIG. 43 is a diagram showing a connecting step using the clip unit 100G.

The operator loads the applicator 200 with the second clip unit 100G (hereinafter also referred to as “clip unit 100G2”). The operator passes the second arm 212 of the clip 2G of the clip unit 100G2 through the connecting ring 27 of the first arm 211G of the clip unit 100G1.

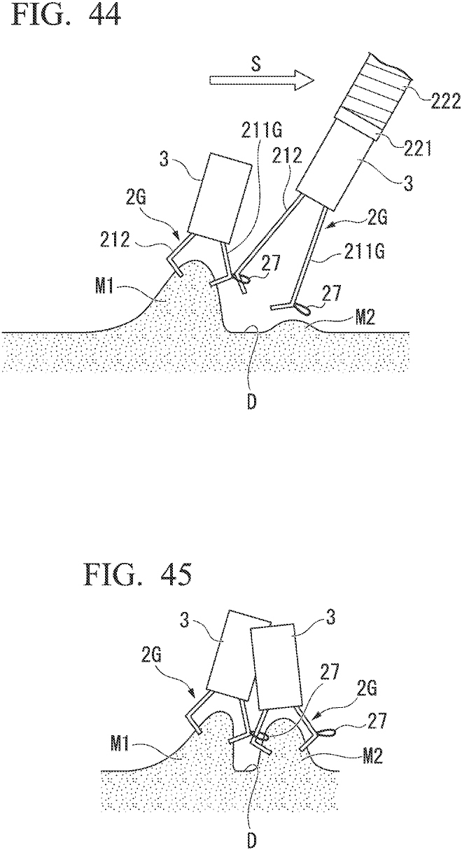

FIG. 44 is a diagram showing the second grasping step using the clip unit 100G.

The operator moves the clip unit 100G along the suturing direction S to move the distal end of the second arm 212 to the vicinity of the second mucous membrane M2. The operator grasps the second mucous membrane M2 with the pair of arms 21G of the clip 2G.

FIG. 45 is a diagram showing the second separation step using the clip unit 100G.

The operator withdraws the sheath 220 and leaves the clip 2G and the holding tube 3 in a state of ligating only the second mucous membrane M2.

According to the clip unit 100G of the present embodiment, the treatment of suturing the defect D can be preferably performed. The clip unit 100G can securely grasp the first mucous membrane M1 and the second mucous membrane M2 by connecting the clips 2G of the two clip units 100G.

The seventh embodiment of the present invention has been described in detail above with reference to the drawings, but the specific configuration is not limited to this embodiment, and design changes and the like are included within the scope of the present invention. Also, the constituent elements shown in the above-described embodiment and modifications shown below can be combined as appropriate.

### Eighth Embodiment

A clip system 300H according to an eighth embodiment of the present invention will be described with reference to FIGS. 46 to 53. In the following description, the same reference numerals are given to the same configurations as those already described, and redundant descriptions will be omitted.

The clip system 300H includes a clip unit 100H and an applicator 200H. The clip unit 100H is loaded into an applicator 200H that can be inserted through a channel of an endoscope and introduced to a treatment position.

FIG. 46 is a diagram showing a clip unit 100H and an applicator 200H according to this embodiment. The applicator 200H includes a first slider 242H and a second slider 243H. The clip unit 100H includes a first arm 211H and a second arm 212H.

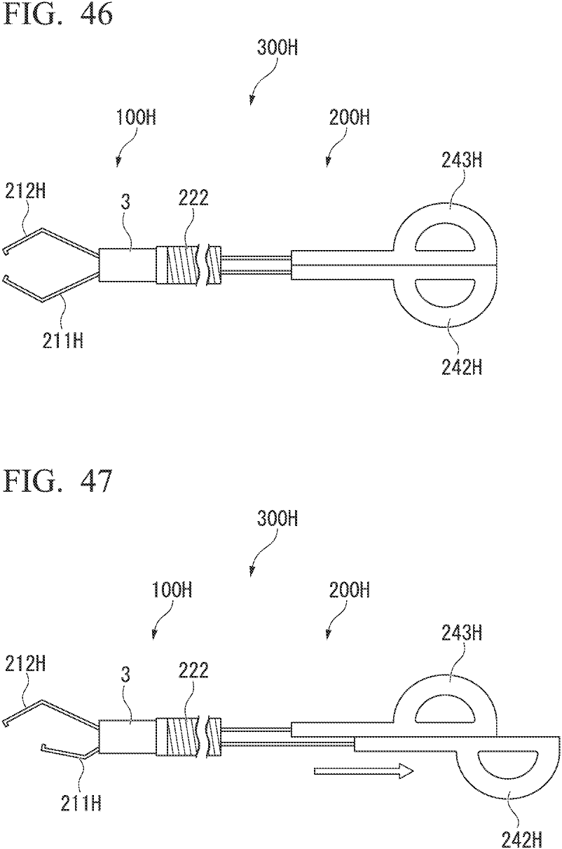

FIG. 47 is a diagram showing the motion of the first arm 211H and the second arm 212H.

The first arm 211H and the second arm 212H are driven independently. The first arm 211H is driven by the first slider 242H. The second arm 212H is driven by a second slider 243H.

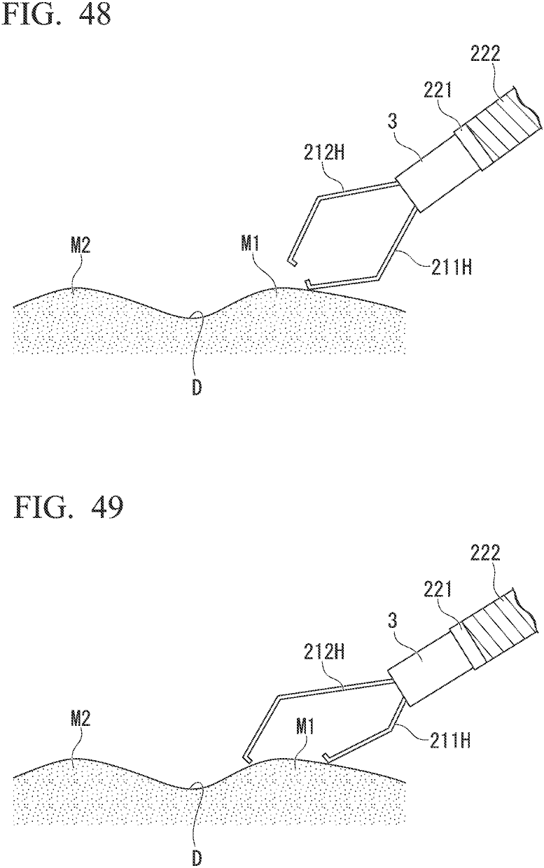

FIG. 48 is a diagram showing approach steps using the clip unit 100H.

The operator approaches the clip unit 100H with the tangential approach AP2 to the defect D. which is the treatment area.

FIG. 49 is a diagram showing a grasping step using the clip unit 100H.

The operator adjusts the lengths of the first arm 211H and the second arm 212H (the amount of protrusion from the holding tube 3). Specifically, the operator adjusts the distal ends of the two arms so that both are in contact with the mucosa. More preferably, the operator brings the first arm 211H into contact with the first mucous membrane M1 and the second arm 212H into contact with the second mucous membrane M2.

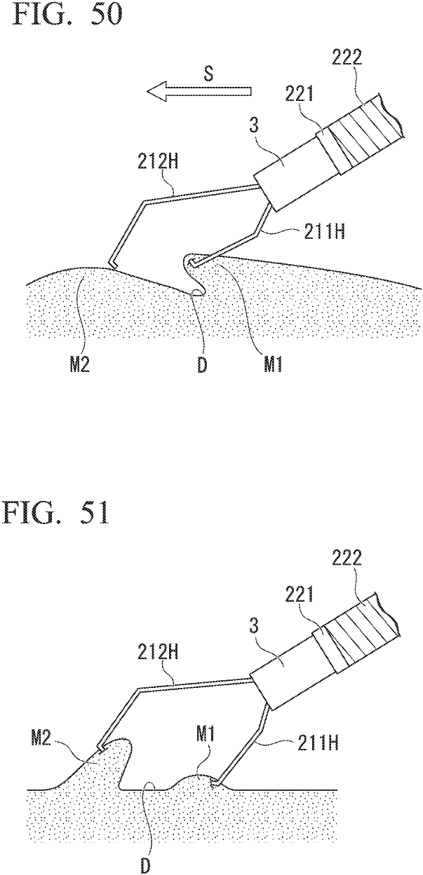

FIG. 50 is a diagram showing a grasping step using the clip unit 100H.

It is preferable that, in a state where the first arm 211H is in contact with the first mucous membrane M1, the operator move the clip unit 100H in the suturing direction S, so that the second arm 212H is brought into contact with the second mucous membrane M2.

FIG. 51 is a diagram showing a pulling step using the clip unit 100H.

The operator retracts the first slider 242H and the second slider 243H to pull the first arm 211H and the second arm 212H. The first arm 211H and the second arm 212H are drawn together to draw the first mucous membrane M1 and the second mucous membrane M2.

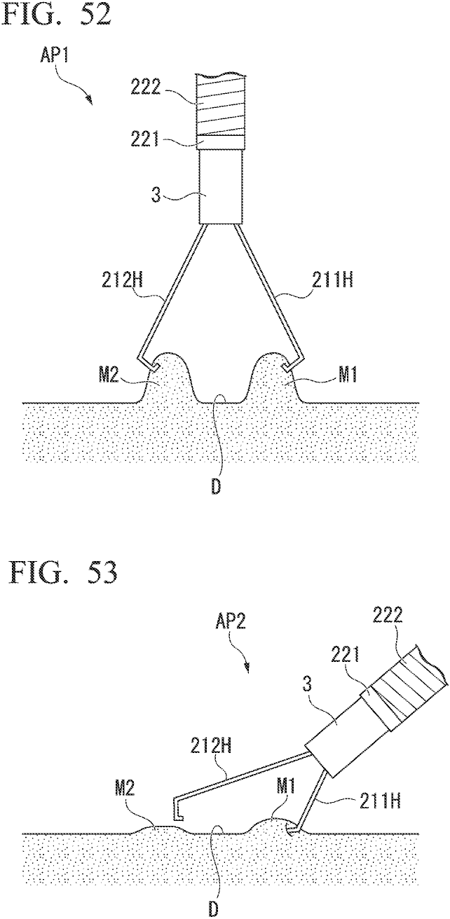

According to the clip unit 100H of the present embodiment, the treatment of suturing the defect D can be preferably performed. In the front approach AP1, by making the length of the first arm 211H and the length of the second arm 212H the same as shown in FIG. 52, the clip unit 100H can reliably grasp the first mucous membrane M1 and the second mucous membrane M2. In the tangential approach AP2, by making the length of the first arm 211H and the length of the second arm 212H different depending on the situation as shown in FIG. 53. the clip unit 100H can reliably grasp the first mucous membrane M1 and the second mucous membrane M2.

The eighth embodiment of the present invention has been described in detail above with reference to the drawings, but the specific configuration is not limited to this embodiment, and design changes and the like are included within the scope of the present invention. Also, the constituent elements shown in the above-described embodiment and modifications shown below can be combined as appropriate.

What is claimed is:

1. A clip system, comprising:
a clip unit having a first arm and a second arm capable of opening and closing;
a wire capable of opening and closing the first arm and the second arm; and
a sheath through which the wire can be inserted,
wherein the clip unit includes a support portion that swingably supports the first arm and the second arm.

2. The clip system according to claim 1, wherein a plane on which the first arm and the second arm swing substantially coincides with a plane on which the first arm and the second arm open and close.

3. The clip system according to claim 1, wherein the support portion can make the first arm swing in a direction to bring the first arm closer to the sheath, and make the second arm swing in a direction away from the sheath.

4. The clip system according to claim 1, wherein
the first arm and the second arm are plate-shaped, and
a plate thickness direction of the first arm and the second arm substantially coincides with a swing direction of the first arm and the second arm.

5. The clip system according to claim 1, wherein
the first arm and the second arm are plate-shaped, and
a plate thickness direction of the first arm and the second arm substantially coincides with an opening and closing direction of the first arm and the second arm.

6. The clip system according to claim 1, wherein the sheath includes a restriction portion that restricts deflection of the sheath in a direction in which the first arm and the second arm swing.

7. The clip system according to claim 6, wherein
the sheath is a coil sheath, and
the restriction portion is a portion welded to an outer peripheral surface of the coil sheath.

8. The clip system according to claim 1, wherein the support portion makes the first arm and the second arm swing when at least one of the first arm and the second arm comes into contact with living tissue.

9. The clip system according to claim 1, wherein the support portion makes the first arm and the second arm swing when an external force is applied to at least one of the first arm and the second arm.
